(19) United States
(12) Patent Application Publication
(10) Pub. No.: US 2012/0225456 A1
(43) Pub. Date: Sep. 6, 2012

(54) METHOD FOR CONDUCTING MULTIPLE REACTIONS IN A SINGLE REACTION TUBE

(75) Inventors: Lars Thomsen, Ipoh (MY); Johnson Kian Kok Ng, Singapore (SG)

(73) Assignee: BIOCHIP DEVISES PTE LTD., Singapore (SG)

(21) Appl. No.: 13/378,524

(22) PCT Filed: Jun. 16, 2010
(86) PCT No.: PCT/SG2010/000227

§ 371 Date: May 18, 2012

(30) Foreign Application Priority Data
Jun. 16, 2009 (GB) 0910291.4

(51) Int. Cl. C12P 19/34; C07H 1/06; C12M 1/34; C12M 1/24; G01N 21/75; B01L 3/14

(52) U.S. Cl. 435/91.2; 422/82.05; 422/549; 435/287.2; 435/304.1; 536/25.41

(57) ABSTRACT

There is disclosed a method for conducting at least two reactions in a reaction tube, said method comprising the steps of providing at least two reaction phases within said reaction tube for allowing said reactions to occur therein, providing a separation phase that is immiscible with said two reaction phases and which is disposed therebetween, providing at least one particle capable of being coupled to a chemical species, wherein said particle is movable between said reaction phases to introduce said chemical species thereto.

### TECHNICAL FIELD

The present invention generally relates to a method for conducting more than one reaction in a single reaction tube.

### BACKGROUND

Over past decades molecular biologists have learned to characterize, isolate and manipulate the molecular components of cells and organisms. These components include deoxyribonucleic acid (DNA), the repository of genetic information, ribonucleic acid (RNA). RNA is a close relative of DNA whose functions range from serving as a temporary working copy of DNA to actual structural and enzymatic functions as well as a functional and structural part of the translational apparatus and proteins, which are the major structural and enzymatic molecules of cells.

Most of the bench-top experiments conducted by molecular biologists to characterize, isolate and manipulate these components are conducted in test tubes, and typically only one single reaction is conducted within each test tube. However, it is required to repeat such reactions, to ensure the accuracy and reliability of the test results as they are subject to experimental errors. The more replicates, the more accurate and reliable the results will be. However variations in the results between test tubes can be significant.

In the recent years, molecular biologists have explored the possibility of conducting more than one reaction in a single test tube. For example, in the field of polymerase chain reactions (PCR), multiplex reactions using molecular beacons or probes have been developed so that the amplification of more than one target gene can be conducted at the same time within a single test tube. However, it is still necessary to replicate these multiplex experiments.

Furthermore, one problem with the conventional PCR techniques is that they are typically conducted on heating blocks which may not have a uniform temperature control within the block. For example, the centre of the heating block may be heated more quickly and to a higher temperature than the edges of the blocks. Accordingly, the various test tubes positioned at the different positions within the heating block are exposed to the different conditions, thereby resulting in undesirable experimental variations in the results obtained from the various test tubes. Such variations also greatly affect the accuracy and reliability of the positive and negative controls.

Other molecular biology experiments such as DNA extraction or purification techniques comprise of a number of steps. For example, the most standard protocol to perform DNA extraction is to conduct at least the steps of cell lysis, washing and elution of the extracted DNA in water. Typically, the cell lysis reaction is conducted in a first test tube. The cell lysate is then poured into another test tube for binding, washing and/or elution. Further purification steps may be conducted in yet another test tube. The transferring of the sample from one test tube to the other may result in undesirable loss in the DNA as some of the DNA may be left behind in the previous test tube or may be accidentally spilled during the transfer.

Therefore, there is a need to provide a method for conducting more than one reaction, such as replicates of the PCR reactions or all the steps of DNA extraction and purification or the combination thereof, in a single integrated test tube, that overcomes, or at least ameliorates, one or more of the disadvantages described above.

### SUMMARY

According to a first aspect, there is provided a method for providing at least two reaction zones in a reaction tube, said method comprising the steps of:

providing in said tube at least two zones formed by respective reaction phases;

providing in said tube a separation phase that is immiscible with said reaction phases and which is disposed therebetween to thereby separate said reaction zones; and

providing at least one particle in said tube that is capable of being coupled to a chemical species, wherein said particle is movable between said reaction zones to introduce said chemical species.

Advantageously, the conducting of multiple reactions in a single reaction tube in accordance with the method disclosed herein reduces tube-to-tube variations. It also reduces or eliminates the need for transferring samples from one tube to another tube. Further, it permits all the steps of a reaction to be conducted in a single integrated tube.

Advantageously, the disclosed method can be used to conduct a number of different types of molecular biology techniques.

Advantageously, the reaction phases are fluidly sealed from one another by the separation phase and the movement of the particle does not cause any disturbance in the phase separations. Advantageously, the separation phase is a liquid separation phase, concurrently permitting the segregation of the reaction phases and the movement of the particle between reaction phases, without the need for a change of state.

More advantageously, the particle substantially couples to the chemical species only, which is to be brought across the reaction phases, and no other solutions or materials. That is, substantially no cross-contamination occurs between the reaction phases.

In one embodiment, the reactions are polymerase chain reactions. In another embodiment, the reactions are the steps for conducting DNA extraction and/or purification. In yet another embodiment, the reactions are a combination of DNA extraction and/or purification steps and polymerase chain reactions (PCR).

Advantageously, positive and/or negative control reactions can be conducted within the same reaction tube to enhance the integrity of the reactions occurring therein.

In one embodiment there is provided a method for conducting PCR, said method comprising the steps of:

providing multiple zones formed by respective reaction phases in a tube, each of said reaction zones comprising at least one of a solution selected to amplify a target nucleic acid;

providing in said tube multiple separation phases that are immiscible with said reaction phases, said separation phases being disposed between a pair of reaction zones; and

providing at least one particle in said tube that is capable of being coupled to a chemical species for PCR reaction, wherein said particle is movable between said reaction zones to introduce said chemical species thereto.

According to a second aspect, there is provided a reaction tube comprising:

at least two reaction phases for allowing a reaction to occur therein;

a separation phase that is immiscible with said at least two reaction phases and which is disposed therebetween;

a particle capable of being coupled to a chemical species, wherein said particle is movable between said reaction phases to introduce said chemical species thereto.

According to a third aspect, there is provided a system for conducting at least two reactions within a reaction tube, said system comprising:

at least one of said reaction tube having at least two reaction phases for allowing a reaction to occur therein, a separation phase that is immiscible with the two reaction phases and which is disposed therebetween, and at least one particle capable of being coupled to a chemical species, wherein said particle is movable between said reaction phases to introduce said chemical species thereto; and

at least one centrifugal means for enabling the movement of said particle between said reaction phases under the action of centrifugal forces.

According to a fourth aspect, there is provided a system for conducting at least two reactions within a reaction tube, said system comprising:

at least one of said reaction tube having at least two reaction phases for allowing a reaction to occur therein, a separation phase that is immiscible with the two reaction phases and which is disposed therebetween, and at least one magnetic particle capable of being coupled to a chemical species, wherein said magnetic particle is movable between said reaction phases to introduce said chemical species thereto; and

at least one magnetic means on the exterior of said reaction tube, wherein said magnetic means is movable along a longitudinal axis substantially parallel to the longitudinal axis of said reaction tube.

### DEFINITIONS

The following words and terms used herein shall have the meaning indicated:

The term “coupled” in reference to a particle being “coupled” to a chemical species includes both direct and indirect physical and chemical bonding between a particle and a chemical species. Chemical bonding covers both covalent and noncovalent bonding of the molecules of a chemical species and includes specifically, but not exclusively, covalent bonding, electrostatic bonding, hydrogen bonding and van der Waals' bonding. Chemical bonding may cover direct chemical bonding in which the molecules of a chemical species form bonds with the particle and indirect chemical bonding in which the molecules of a chemical species form bonds with another chemical species that in turn bonds to the particle. Physical bonding refers to any attractive, nonchemical interaction which is able to hold a chemical species on the surface of the particle. The physical bonding may also be direct and indirect in that for direct physical bonding, the chemical species is physically bonded directly to the particle while in indirect physical bonding, the chemical species is bonded directly to another chemical species which is physically bonded to the particle.

The term “chemical species” is to be interpreted broadly to include any entity such as atoms, molecules, molecular fragments and ions. The entity may be a biological sample, a non-biological sample or nucleic acid.

A “biological sample” may be selected from the group consisting of dermal swabs, cerebrospinal fluid, blood, sputum, bronchio-alveolar lavage, bronchial aspirates, lung tissue, and urine. A “non-biological sample” may be a liquid suspension comprising powders, particles from air samples, and particles from earth samples and surface swipes. The biological and non-biological samples may be cultured to facilitate the evaluation of the presence of a microorganism for example, such as B. anthracis.

The term “nucleic acid” may include, for example, but is not limited to deoxyribonucleic acid (DNA), ribonucleic acid (RNA), and artificial nucleic acids such as peptide nucleic acid (PNA), morpholino and locked nucleic acid (LNA), glycol nucleic acid (GNA) and threose nucleic acid (TNA). In the present context, the term “nucleic acid”, “nucleic acid sequence” or “nucleic acid molecule” should be interpreted broadly and may for example be an oligomer or polymer of ribonucleic acid (RNA) or deoxyribonucleic acid (DNA) or mimetics thereof. This term includes molecules composed of naturally-occurring nucleobases, sugars and covalent internucleoside (backbone) linkages as well as molecules having non-naturally occurring nucleobases, sugars and covalent internucleoside (backbone) linkages which function similarly or combinations thereof. Such modified or substituted nucleic acids may be preferred over native forms because of desirable properties such as, for example, enhanced affinity for nucleic acid target molecule and increased stability in the presence of nucleases and other enzymes, and are in the present context described by the terms “nucleic acid analogues” or “nucleic acid mimics”. Preferred examples of nucleic acid mimetics are peptide nucleic acid (PNA-), Locked Nucleic Acid (LNA-), xylo-LNA-, phosphorothioate-, 2′-methoxy-, 2′-methoxyethoxy-, morpholino- and phosphoramidate-comprising molecules or functionally similar nucleic acid derivatives.

The term “particle” is to be interpreted broadly to include any magnetic or non-magnetic particle of any shape, that is capable of being coupled to a chemical species. The particle may be a magnetic particle that is substantially spherical in shape.

The term “magnetic means” is to be interpreted broadly to include any material that is capable of exerting attractive or repulsive forces on a body by way of exerting a magnetic field over the body. Exemplary examples of a material suitable for use as a magnetic means are nickel, iron, cobalt, gadolinium and their alloys.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Disclosure of Optional Embodiments

Exemplary, non-limiting embodiments of a method for conducting more than one reaction in a reaction tube, will now be disclosed.

The reaction tube may contain a plurality of the particles. The particles may be moveable across the reaction phases under the action of centrifugal or gravitational forces.

In one embodiment, the particles are magnetic particles, which are then moveable across the reaction phases under the action of a magnetic force.

The disclosed method may then further comprise the step of providing at least one magnetic means on the exterior of the reaction tube, wherein the magnetic means is movable along a longitudinal axis substantially parallel to the longitudinal axis of the reaction tube.

In one embodiment, the reaction phases are aqueous phases.

In one embodiment, the immiscible phase is an oil phase. The oil phase may comprise mineral oil.

In one embodiment, the reactions comprise of nucleic acid extraction reactions.

In one embodiment, the reactions comprise of polymerase chain reactions.

The aqueous phases may comprise of a cell lysis solution, a washing buffer, an elution buffer or a polymerase chain reaction solution.

In one embodiment, the magnetic particle is spherical in shape and has a diameter in the range of about 0.5 microns to about 300 microns, or about 0.5 microns to about 100 microns, or about 0.5 microns to about 50 microns, or about 0.5 microns to about 25 microns, or about 0.5 microns to about 10 microns, or about 0.5 microns to about 5 microns, or about 1 micron.

The magnetic beads used may be Magprep® silica beads (Merck, France). In one embodiment, the amount of Magprep® silica beads used is in the range of about 10 μg to about 200 μg, about 20 μg to about 175 μg, about 30 μg to about 150 μg, about 40 μg to about 125 μg, about 50 μg to about 100 μg.

In one embodiment, the reaction tube is a capillary tube. The capillary tube may be about 35 mm in length with an inner diameter of about 0.75 mm and the outer diameter of about 1 mm.

In one embodiment, the capillary is a glass capillary. The glass capillary may be made of borosilicate glass or any other material that has excellent thermal properties and can withstand the rapid changes in temperature with breaking.

The magnetic means may be moving at varying or constant speed, or a combination thereof. The amount of magnetic beads used is taken into consideration to determine the speed of the movement of the magnetic means such as to control the movement of the magnetic beads within the reaction tube.

The lower the speed of movement of the magnetic means, the higher is the magnetic force holding the magnetic beads close to one another to form a cluster. This provides sufficient momentum for moving the magnetic beads across the aqueous-oil interface, i.e. from an aqueous phase across to an oil phase, and vice versa.

The higher the amount of magnetic beads used, the lower the speed of movement of the magnetic means is required for the magnetic beads to form a cluster with sufficient momentum to move across the aqueous-oil interface.

The magnetic means may be moving at a speed in the range of about 0.1 mm/s to about 10 mm/s, about 1 mm/s to about 9 mm/s, about 2 mm/s to about 8 mm/s, about 3 mm/s to about 7 mm/s, about 4 mm/s to about 6 mm/s.

The polymerase chain reactions may include fluorescence dyes that may aid in the detection of the presence and/or amount of the nucleic acid present before, during and/or after completion of the reactions. The polymerase chain reactions may also be multiplex reactions. The fluorescence dyes employed may be SYBR Green, or molecular probes or beacons.

In one embodiment, the disclosed system further comprises an optical detection means for detecting the presence and/or amount of nucleic acid in each of said immiscible phases.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a schematic diagram of a glass capillary comprising more than one immiscible phases.

FIG. 2 is a schematic diagram of a reaction system comprising the glass capillary comprising more than one immiscible phases as shown in FIG. 1.

FIGS. 3a and 3b are schematic diagrams of a section of the glass capillary comprising more than one immiscible phases as shown in FIG. 1 to illustrate the movement of magnetic beads contained therein.

### DETAILED DISCLOSURE OF EMBODIMENTS

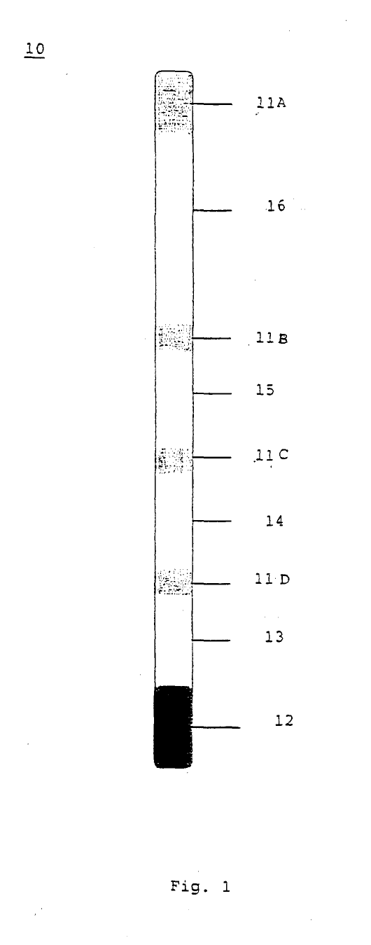

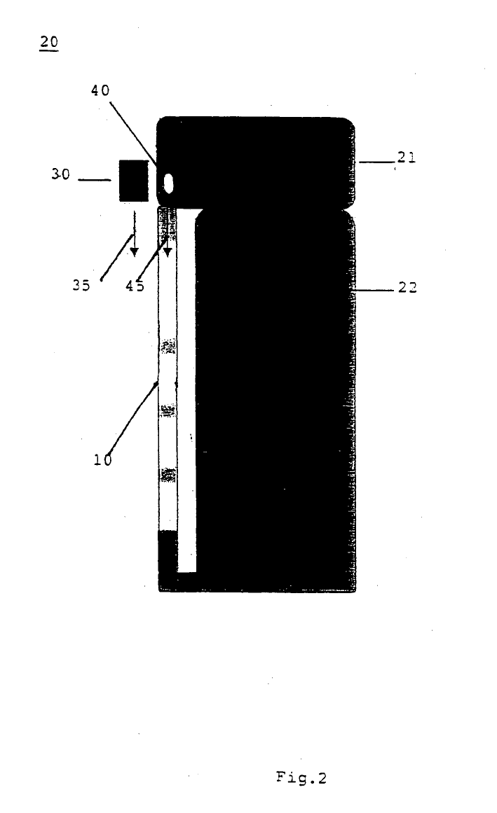

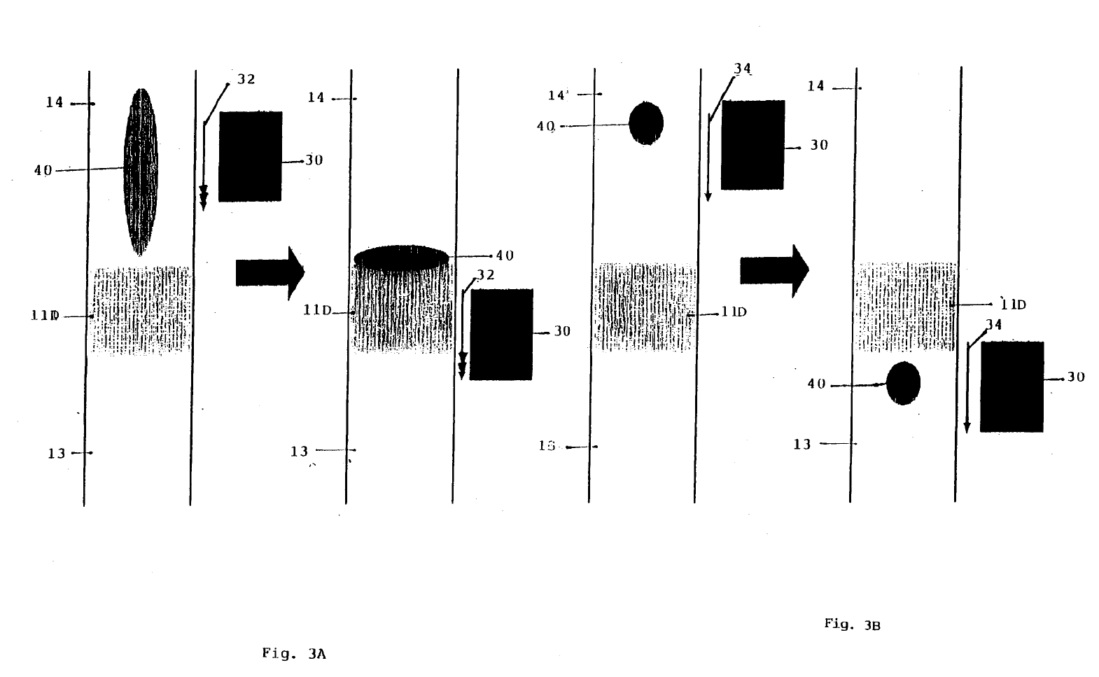

FIG. 1 shows a glass capillary 10 having four separation phases in the form of mineral oil layers (11A, 11B, 11C, 11D) and four reaction zones formed by aqueous layers consisting of a control solution 13, a first test solution 14, a second test solution 15 and a wash solution 16 contained therein. The control solution 13, the first test solution 14 and the second test solution 15 are solutions containing the necessary reagents for conducting polymerase chain reactions (PCRs). When in use, the first test solution 14 and the second test solution 15 provide for the conducting of the same reaction independently and in duplicates. The glass capillary 10 also contains at its bottom end an epoxy seal 12 to seal the bottom of the glass capillary 10, to thereby prevent leakage of the solutions and mineral oil contained therein. The function of the glass capillary 10 will be further described below with reference to FIGS. 2, 3A and 3B.

FIG. 2 shows a reaction system 20 having a glass capillary 10 as shown in FIG. 1 and with contents as described above. The reaction system 20 also comprises of a sample holder 21, a capillary holder 22 and a magnet 30. Magnetic beads 40 that are encoded with DNA are first contained within the sample holder 21. The magnet 30 provides a magnetic force for moving the magnetic beads 40 along the glass capillary 10. When in operation, as the magnet 30 moves, downwards in the direction as shown by arrow 35, the magnetic beads 40 are moved downwards in the direction as shown by arrow 45. This allows for the transfer and mixing of the DNA encoded on the magnetic beads 40 across the mineral oil layers (11A, 11B, 11C, 11D) and within the various aqueous layers (13, 14, 15, 16) in accordance with experimental requirements, wherein the control solution 13 is a positive control, which may be used to check the integrity of the PCR solutions contained therein.

In one embodiment, the control solution 13 functions as a negative control. Accordingly, it is not necessary to move the magnetic beads 40 across the mineral oil layer 11D to the control solution 13.

FIGS. 3A and 3B show a section of the glass capillary 10 to facilitate the illustration of the movement of the magnetic beads 40 along the glass capillary 10. In comparison, the magnet 30 as shown in FIG. 3A is moved at a faster speed (downwards movement as shown by arrow 32) than that as in FIG. 3B (downwards movement as shown by arrow 34).

Referring to FIG. 3A, the magnet 30 is moved at a speed of 5 mm/s. Due to a faster speed of movement of the magnet 30, the magnetic beads 40 in the second test solution 14 are dispersed and therefore do not have sufficient momentum to pass through the mineral oil layer 11D. This operation is desirable, for example, when the control solution 13 functions as a negative control.

On the other hand, referring to FIG. 3B, the magnet is moved at a speed of 3 mm/s. Due to a slower speed of movement of the magnet 30, the magnetic beads 40 in the second test solution 14 are tightly clumped together to thereby provide sufficient momentum for the magnetic beads 40 to pass through the mineral oil layer 11D. This operation is desirable, for example, when the control solution 13 functions as a positive control.

### APPLICATIONS

It will be appreciated that the disclosed method allows for conducting of more than one physical or chemical reaction in a single reaction tube.

The disclosed method allows for the conducting of the same experiment in replicates, and yet avoids test tube to test tube variations which may significantly affects the accuracy and reliability of experimental results. Advantageously, positive and/or negative control experiments can also be included in the same reaction tube to further enhance the integrity of the experiments conducted therein.

It will be appreciated that the disclosed methods can be used to conduct a number of different types of molecular biology experiments, such as polymerase chain reactions, DNA extraction and/or DNA purification. Advantageously, the disclosed method also eliminates the need to transfer experimental samples or reactions from one test tube to another.

Advantageously, the magnetic beads are coupled to the chemical species of interest for conducting of the reactions. The movement of the magnetic beads also aid in the mixing of the chemical species within the reaction phases. More advantageously, it will be appreciated that the movement of the magnetic beads across the reaction and separation phases does not does not cause any disturbance in the phase separations.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method for providing at least two reaction zones in a reaction tube, said method comprising the steps of: providing in said tube at least two zones formed by respective reaction phases; providing in said tube a separation phase that is immiscible with said reaction phases and which is disposed therebetween to thereby separate said reaction zones; and providing at least one particle in said tube that is capable of being coupled to a species, wherein said particle is movable between said reaction zones to introduce said species.

2. The method as claimed in claim 1, wherein said particle is movable under the action of centrifugal or gravitational forces.

3. The method as claimed in claim 1, wherein said particle is a magnetic particle.

4. The method as claimed in claim 3, further comprising the step of providing at least one magnetic means on the exterior of said reaction tube, wherein said magnetic means is movable along a longitudinal axis substantially parallel to a longitudinal axis of said reaction tube.

5. The method as claimed in claim 1, wherein said reaction phases are aqueous phases.

6. The method as claimed in claim 1, wherein said immiscible phase is an oil phase that is immiscible with said reaction phases.

7. The method as claimed in claim 1, wherein said reactions are independently chemical or physical reactions.

8. The method as claimed in claim 7, wherein said reactions include nucleic acid extraction reactions.

9. The method as claimed in claim 7, wherein said reactions include polymerase chain reactions.

10. The method as claimed in claim 5, wherein said aqueous phases comprise at least one of: (i) a cell lysis solution; (ii) a washing buffer; (iii) an elution buffer and (iv) a polymerase chain reaction solution.

11. The method as claimed in claim 6, wherein said oil phase comprises mineral oil.

12. The method as claimed in claim 3, wherein said magnetic particle is substantially spherical in shape.

13. The method as claimed in claim 12, wherein said spherical magnetic particle has a diameter that is in the micron range.

14. The method as claimed in claim 3, wherein plural magnetic particles are provided in said tube.

15. The method as claimed in claim 14 when dependent on claim 4, wherein said magnetic means is configured to move said plural magnetic particles at a speed between said two reaction zones such that said plural magnetic particles pass from one reaction zone to another as a substantially single cluster.

16. The method as claimed in claim 4, wherein said magnetic means comprises a magnet that is made of a metal selected from the group consisting of nickel, iron, cobalt, gadolinium and alloys thereof.

17. A reaction tube comprising: at least two reaction phases for allowing a reaction to occur therein; a separation phase that is immiscible with said at least two reaction phases and which is disposed therebetween; and a particle capable of being coupled to a chemical species, wherein said particle is movable between said reaction phases to introduce said chemical species thereto.

18. The tube as claimed in claim 17, wherein said particle is a magnetic particle.

19. The tube as claimed in claim 17, wherein said reaction tube is a glass capillary.

20. The tube as claimed in claim 19, wherein said glass capillary is about 20 mm to about 50 mm in length.

21. The tube as claimed in claim 19, wherein said glass capillary has an inner diameter of about 0.5 mm.

22. A system for conducting at least two reactions within a reaction tube, said system comprising: at least one of said reaction tube having at least two reaction phases for allowing a reaction to occur therein, a separation phase that is immiscible with the two reaction phases and which is disposed therebetween, and at least one particle capable of being coupled to a chemical species, wherein said particle is movable between said reaction phases to introduce said chemical species thereto; and at least one centrifugal means for enabling the movement of said particle between said reaction phases under the action of centrifugal forces.

23. A system for conducting at least two reactions within a reaction tube, said system comprising: at least one of said reaction tube having at least two reaction phases for allowing a reaction to occur therein, a separation phase that is immiscible with the two reaction phases and which is disposed therebetween, and at least one magnetic particle capable of being coupled to a chemical species, wherein said magnetic particle is movable between said reaction phases to introduce said chemical species thereto; and at least one magnetic means on the exterior of said reaction tube, wherein said magnetic means is movable along a longitudinal axis substantially parallel to the longitudinal axis of said reaction tube.

24. The system as claimed in claim 23, further comprising an optical detection means for detecting the presence and/or amount of said chemical species in said reaction phases.

25. A method for conducting polymerase chain reaction (PCR), said method comprising the steps of: providing multiple zones formed by respective reaction phases in a tube, each of said reaction zones comprising at least one of a solution selected to amplify a target nucleic acid; providing in said tube multiple separation phases that are immiscible with said reaction phases, said separation phases being disposed between a pair of reaction zones; and providing at least one particle in said tube that is capable of being coupled to a chemical species for PCR reaction, wherein said particle is movable between said reaction zones to introduce said chemical species thereto.

26. The method as claimed in claim 25, wherein said solutions of said plural reaction zones are selected to amplify different target nucleic acids.

27. The method as claimed in claim 25, wherein said particle is movable under the action of centrifugal or gravitational forces.

28. The method as claimed in claim 25, wherein said particle is a magnetic particle and is movable under the action of a magnetic force.

29. The method as claimed in claim 5, wherein plural magnetic particles are provided in said tube.

30. The method as claimed in claim 6, wherein plural magnetic particles are provided in said tube.

31. The method as claimed in claim 7, wherein plural magnetic particles are provided in said tube.
